(19) United States
(12) Patent Application Publication
(10) Pub. No.: US 2006/0207603 A1
(43) Pub. Date: Sep. 21, 2006

(54) Ventilator attachment for tracheal T-tubes

(75) Inventors: Robert R. Lorenz, Shaker Heights, OH (US); Isaac Eliachar, Indian Wells, CA (US); Sudish Murthy, Cleveland Heights, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 11/352,960

(22) Filed: Feb. 11, 2006

Related U.S. Application Data
(60) Provisional application No. 60/652,651, filed on Feb. 14, 2005.

(51) Int. Cl. A61M 16/00; A62B 9/06

(52) U.S. Cl. 128/207.14; 128/200.26; 128/207.15; 128/207.16

(57) ABSTRACT

An apparatus for use with a tracheal T-tube having transversely extending first and second passages that communicate with each other. The apparatus has a body with a generally tubular shape and a channel extending between distal and proximal ends. The body has cylindrical inner and outer walls that define a wall thickness. The body can be inserted into the first passage so that the distal end lies in the second passage. The apparatus has an inflatable device operatively secured to its distal end, and means for selectively inflating and deflating the inflatable device. The body also has a tube which defines a lumen extending between the ends and fluidly connecting the inflatable device with the means for selectively inflating and deflating the inflatable device.

### RELATED APPLICATION

This application claims priority from U.S. provisional patent application Ser. No. 60/652,651, filed on Feb. 14, 2005, the subject matter of which is incorporated herein by reference.

### TECHNICAL FIELD

The present invention relates generally to medical devices, and more particularly relates to ventilator attachments for tracheal T-tubes.

### BACKGROUND

There are many indications for tracheostomy, in which a device known as a tracheostomy tube is inserted into the trachea for inhalation purposes. The tracheostomy tube is typically a curved plastic tube fitted adjacent its distal end (which is inserted downwardly into the trachea through an incision) with an external balloon or cuff element which can be selectively inflated within the trachea to retain the tube in place and allow for mechanical ventilation. With the tracheostomy tube in place, the outer tube end is operatively connected to a ventilator which functions to cyclically force air downwardly through the inserted tube into the tracheal passage portion below it, and into the patient's lungs.

Conventional balloon-tipped tracheostomy tubes can cause interior tracheal scarring which restricts the tracheal passage. This scarring typically arises due to the fact that the curved tracheostomy tube does not conform to the essentially straight tracheal anatomy. As a result, pressure forces imposed on the trachea's interior surface can lead to pressure necrosis of the tracheal wall.

Upon removal of the tracheostomy tube, this interior scarring, which tends to restrict the trachea, can necessitate the subsequent insertion into the trachea of a device known as a T-tube stent, or a “Montgomery tube”, which functions to hold the scarred and restricted tracheal portion open and prevent it from unduly restricting patient breathing.

While the subsequent use of conventional T-tube stents in this manner is quite beneficial in holding open scar tissue-restricted tracheal passage areas during patient recuperation, it does not permit the use of a ventilator to assist the patient's breathing. Air forced into the outwardly projecting transverse stent portion cannot be effectively forced downward into the patient's lungs because both the upper and lower ends of the inserted T-tube body within the trachea are open, and air passes preferentially out of the mouth because of less resistance.

Consequently, various methods have been developed for ventilating patients with indwelling T-tubes. In one known method, the patient's oropharynx is packed with a blocking material such as gauze or polyurethane. This method, however, is prone to several drawbacks including air leakage, dislodgment, and pressure necrosis of the tissues. Furthermore, this method is typically uncomfortable for the conscious patient and only suitable for temporary ventilation.

In a second known method, a small endotracheal tube is first inserted through the T-tube's horizontal limb and then into the inferior limb of the vertical segment. To do so, the endotracheal tube must be smaller than the T-tube diameter to allow for insertion and removal. As a result, however, this method presents several limitations. First, as the endotracheal tube is not designed for this function, an incomplete seal is formed between the T-tube and the endotracheal tube and a faulty air flow circuit results. Second, the 90-degree turn of the T-tube can cause undesirable kinking of the endotracheal tube. Finally, the endotracheal tube can be inserted too far into the trachea (e.g., right mainstem intubation) as there is no physical means for guiding the endotracheal tube to a desired point in the T-tube.

In a third known method, a “Hebeler” T-tube with an inflatable balloon obstructing the superior limb of the T-tube's vertical segment can be used to mechanically ventilate the patient. This type of tube is not suitable for long-term use as the balloon is permanently placed and thus can gather secretions and become infected. And furthermore, patients generally do not care for the constant pressure of external air cuff needed to either inflate or deflate the balloon.

In a fourth known method, a removable balloon on a Fogarty catheter is first placed through the T-tube's horizontal limb and then into the superior vertical limb. The loose-fitting catheter is prone to dislodgment and is unwieldy since the attachment to the ventilator machine can create an air leak in the circuit. Further, non-otolaryngologists can be uncomfortable using this method as placement of the balloon is difficult to approximate correctly.

From the foregoing it can be concluded that conventional methods for mechanically ventilating patients with indwelling T-tubes is, for a variety of reasons, not wholly satisfactory.

### SUMMARY OF THE INVENTION

Accordingly, the present invention provides an improved apparatus for mechanically ventilating patients with indwelling tracheal T-tubes.

In one aspect of the present invention, an apparatus for use with a tracheal T-tube having transversely extending first and second passages that communicate with each other. The apparatus has a body with a generally tubular shape and a channel extending between distal and proximal ends. The body has cylindrical inner and outer walls that define a wall thickness. The body can be inserted into the first passage so that the distal end lies in the second passage. The apparatus has an inflatable device operatively secured to its distal end, and means for selectively inflating and deflating the inflatable device. The body includes a tube which defines a lumen extending between the ends and fluidly connecting the inflatable device with the means for selectively inflating and deflating the inflatable device.

In another aspect of the present invention, an apparatus is provided which comprises a tracheal T-tube having transversely extending first and second passages that communicate with each other, and a body having a generally tubular shape with a channel extending between distal and proximal ends. The body has cylindrical inner and outer walls that define a wall thickness. The body can be disposed in the first passage of the T-tube so that its distal end lies in the second passage of the T-tube. The apparatus has an inflatable device operatively secured to the distal end of the body, and a means for selectively inflating and deflating the inflatable device. The body includes a lumen extending between the ends and fluidly connecting the inflatable device with the means for selectively inflating and deflating the inflatable device.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a perspective view showing an apparatus for mechanically ventilating a patient with a tracheal T-tube and a ventilator attachment for the tracheal T-tube;

FIG. 2 is an enlarged perspective view taken from the proximal end of the apparatus;

FIG. 3 is an enlarged scale cross-sectional view of the apparatus inserted into a tracheal T-tube;

FIG. 4 is an alternate embodiment of the apparatus of FIG. 1;

FIG. 5 is a cross-sectional view of the apparatus inserted into an indwelling tracheal T-tube in a patient's trachea;

FIG. 6 is another alternative embodiment of the apparatus of FIG. 1;

FIG. 7 is an enlarged side cross-sectional view of the apparatus of FIG. 6 inserted into a tracheal T-tube; and

FIG. 8 is an alternative embodiment of the apparatus of FIG. 6.

### DETAILED DESCRIPTION

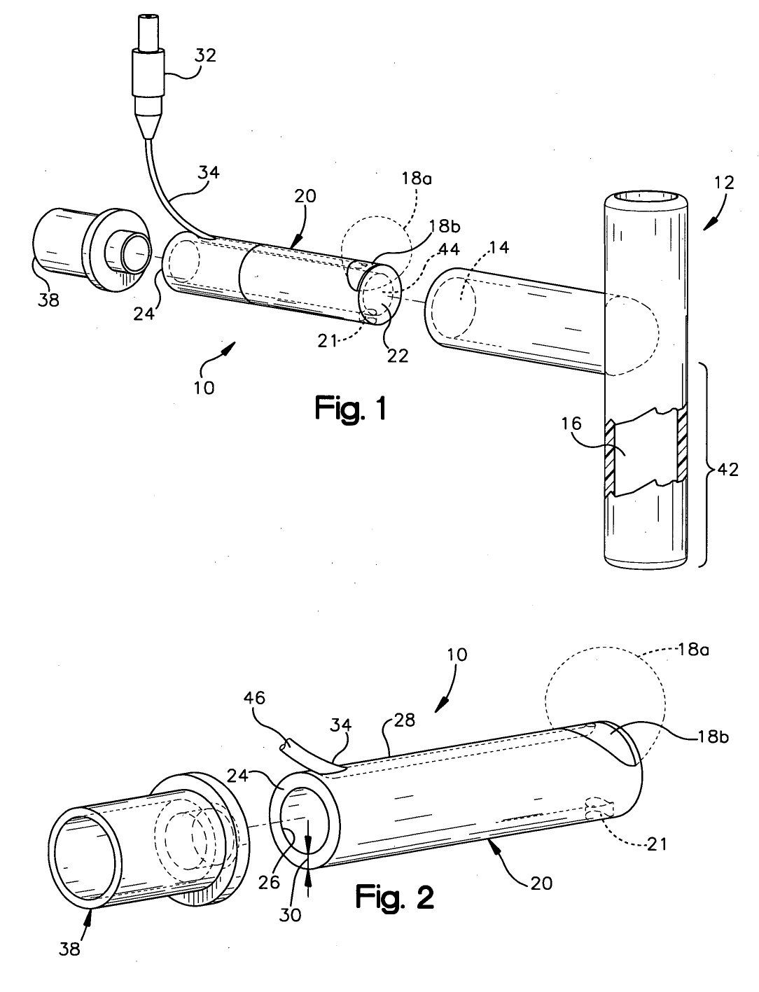

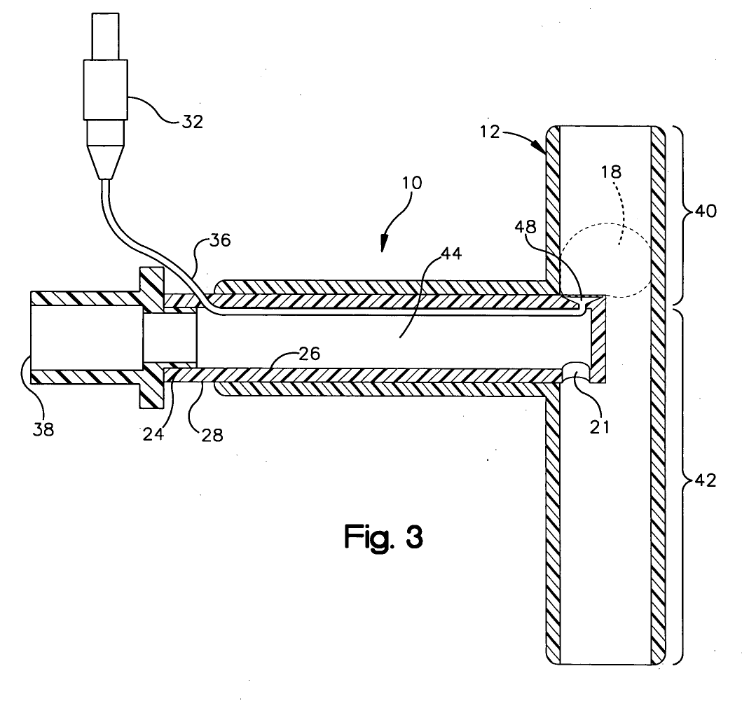

Perspectively illustrated in FIG. 1 is an apparatus 10 for mechanically ventilating a patient with an indwelling tracheal T-tube 12. The apparatus 10 is used with a tracheal T-tube 12 having transversely extending first and second passages 14 and 16 that communicate with each other. The second passage 16 extends between upper and lower portions 40 and 42 (FIG. 3) of the T-tube 12.

The apparatus 10 includes a body 20 with a generally tubular shape and a channel 44 extending between distal and proximal ends 22 and 24. The body 20 has cylindrical inner and outer walls 26 and 28 that define a wall thickness 30. The body 20 can be inserted into the first passage 14 so that the distal end 22 lies in the second passage 16. In this case, the outer wall 28 of the body 20 fits snugly against the inner wall of the T-tube 12. The body 20 includes a tube 34 that defines a lumen 46 (FIG. 2) extending between the distal and proximal ends 22 and 24 and fluidly connecting an inflatable device 18 with a means 32 for selectively inflating and deflating the inflatable device.

As shown in FIG. 1, the distal and proximal ends 22 and 24 respectively include closed and open configurations. Alternatively, the distal end 22 can have an open configuration (not shown) similar to the open configuration of the proximal end 24. The distal end 22 of the body 20 has a planar or straightened shape and includes at least one opening 21 oppositely located from the inflatable device 18. The opening 21 may have a variety of shapes and configurations, including, but not limited to, circular (FIG. 1), rectangular, square, triangular, ovoid, and the like. When the apparatus 10 is inserted into the tracheal T-tube 12, the distal end 22 abuts the wall of the second passage 16 so that a continuous air passage—comprised of the channel 44, the opening 21, and the second passage 16—is formed.

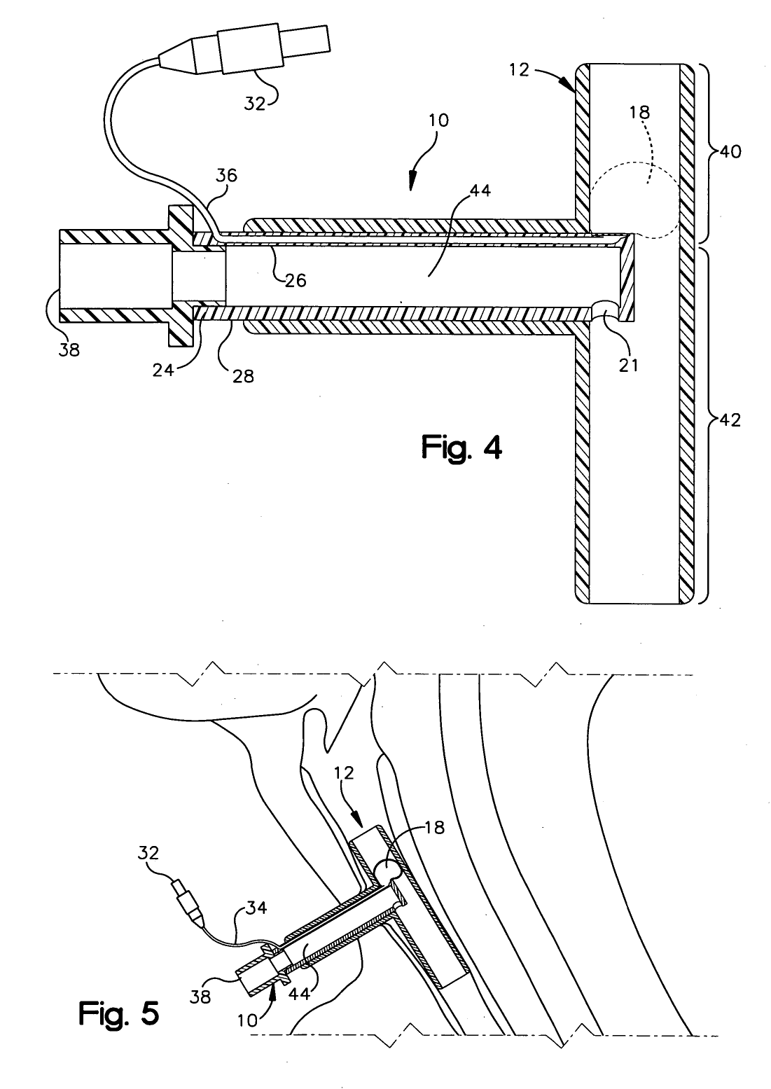

As shown in FIG. 3, the tube 34 in the body 20 extends along the inner wall 26 of the body and includes a transverse portion 36 extending outwardly through the wall thickness 30 intermediate the distal and proximal ends 22 and 24. Alternatively, as shown in FIG. 4, the tube 34 can be disposed within the wall thickness 30 and include a transverse portion 36 extending outwardly through the wall thickness intermediate the distal and proximal ends 22 and 24.

The body 20 and tube 34 can be made from any flexible, resiliently yieldable material which exhibits a substantially smooth, non-adherent surface. Examples of such material can include plastics (e.g., polyacrylics, polyolefins, and vinyl polymers such as polyvinyl chloride) and silicon. The material can also be transparent.

The body 20 can also include a means for correctly orienting the apparatus 10 relative to the T-tube 12. A key-and-channel configuration (not shown) may be used where, for example, the apparatus 10 includes a channel which can be aligned with a key disposed in the first passage 14 of the T-tube 12. Alternatively, the apparatus 10 can include a key which is fitted to a channel disposed in the first passage 14 of the T-tube 12. Another means for correctly orienting the apparatus 10 may include opposing markers (not shown) disposed about the apparatus and the T-tube 12. For instance, a colored marker may be placed on both the apparatus 10 and the T-tube 12. When the apparatus 10 is inserted into the T-tube 12, the markers on both the apparatus and the T-tube are aligned to ensure that the apparatus and the T-tube are properly oriented relative to each other.

As shown in FIG. 3, the inflatable device 18 is operatively secured to the distal end 22 of the body 20. The inflatable device 18 can include any device, such as a balloon, capable of being inflated to a desired volume. The distal end 22 of the body 20 includes a recess 48 in which the inflatable device 18 is disposed when in its deflated position 18_{b} (FIG. 2). The apparatus 10 also includes a means 32 for selectively inflating and deflating the inflatable device 18. The means 32 can include a source of pressurized air, such as an air syringe, for example.

The inflatable device 18 may be inflated by the means 32. By forcing air from the means 32 through the lumen 46, the inflatable device 18 can be inflated 18ₐ to internally occlude the upper portion 40 of the second passage 16 and preclude air movement inwardly or outwardly through the upper portion. Alternatively, air can be evacuated through the lumen 46 to deflate the inflatable device 18 to the position 18_{b} in FIG. 2. In this case, the inflatable device 18 is disposed within the recess 48 in the outer surface of the distal end 22 of the body 20. Withdrawing air from the lumen 46 clears the upper portion 40 of the second passage 16 and permits air flow in either vertical direction therethrough.

FIG. 5 is a cross-sectional illustration showing the apparatus 10 when inserted into an indwelling T-tube 12 in a patient's trachea. In this position, the apparatus 10 can be used as a ventilator fitting by inflating the inflatable device 18 via the lumen 46 and means 32, and then inserting a ventilator adapter 38 into the open proximal end 24 of the apparatus. A mechanical ventilator (not shown) can then be connected to the ventilator adapter 38. Operation of the ventilator cyclically forces air inwardly through the body 20, through the opening 21, downwardly through the lower portion 42 of the second passage 16, and into the patient's lungs. Importantly, since the inflated 18ₐ inflatable device 18 blocks the upper portion 40 of the second passage 16, nearly all of the inflowing air is downwardly directed into the patient's lungs. When the need for mechanical breathing assistance ceases, the inflatable device 18 can be deflated to the position 18_{b} in FIG. 2. Any minor volume lost due to air passing around the ventilator fitting can be compensated for by ventilator adjustments.

In addition, when the distal end 22 of the apparatus 10 is located in the second passage 16, the inflatable device 18 can be deflated 18_{b} and a suitable plug member (not shown) can be inserted into the open proximal end 24 of the apparatus. Insertion of the plug member permits the patient to breathe normally as the upper portion 40 of the second passage 16 is no longer occluded and air can pass upwardly and downwardly through the patient's trachea without escaping from the first passage 14 of the T-tube 12.

Alternatively, when the inflatable device 18 is deflated 18_{b}, the apparatus 10 can be positioned such that the distal end 22 is not positioned within the second passage 16. The plug member can then be inserted into the proximal end 24 of the apparatus 10. With the apparatus 10 in this position, the patient can breathe normally as air can pass upwardly and downwardly through the patient's trachea.

Should the need arise to again provide the patient with mechanical breathing assistance, the apparatus 10 can be rapidly converted to a ventilator fitting by removing the plug member, inserting the ventilator adapter 38 into the open proximal end 24 of the apparatus, and re-inflating the inflatable device 18 to again occlude the upper portion 40 of the second passage 16.

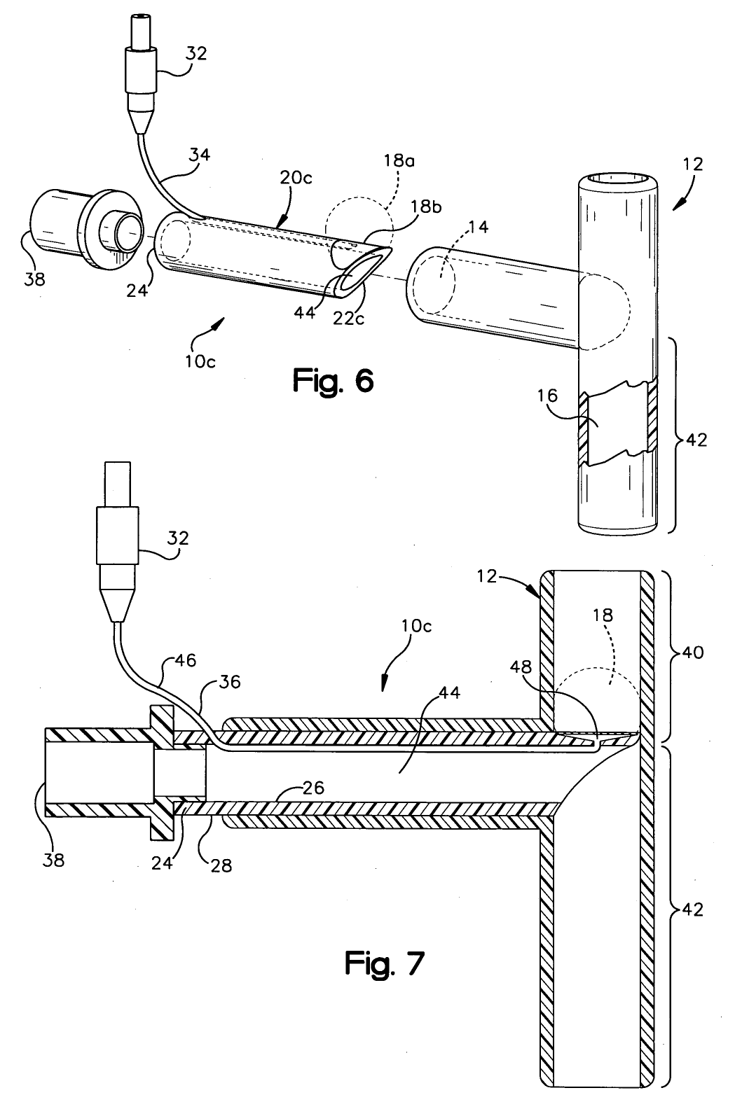

FIGS. 6-8 illustrate an alternative embodiment of the present invention. The apparatus 10_{c} of FIGS. 6-8 is identically constructed as the apparatus 10 of FIGS. 1-5, except where as described below. In FIGS. 6-8, structures that are identical as structures in FIGS. 1-5 use the same reference numbers, whereas structures that are similar but not identical carry the suffix “c”.

As shown in FIG. 6, the apparatus 10C includes a body 20, with a generally tubular shape and a channel 44 extending between distal and proximal ends 22, and 24. The body 20_{c} has cylindrical inner and outer walls 26 and 28 that define a wall thickness 30. The body 20, can be inserted into the first passage 14 so that the distal end 22_{c} lies in the second passage 16. In this case, the outer wall 28 of the body 20_{c} fits snugly against the inner wall of the T-tube 12. The body 20, includes a tube 34 that defines a lumen 46 (FIG. 7) extending between the distal and proximal ends 22_{c} and 24 and fluidly connecting the inflatable device 18 with the means 32 for selectively inflating and deflating the inflatable device.

As illustrated in FIG. 6, the distal and proximal ends 22, and 24 each have an open configuration. The distal end 22, of the body 20_{c} has an angled shape and, when inserted into the T-tube 12, abuts the wall of the second passage 16 so that a continuous fluid passage is formed by the channel 44 and the second passage.

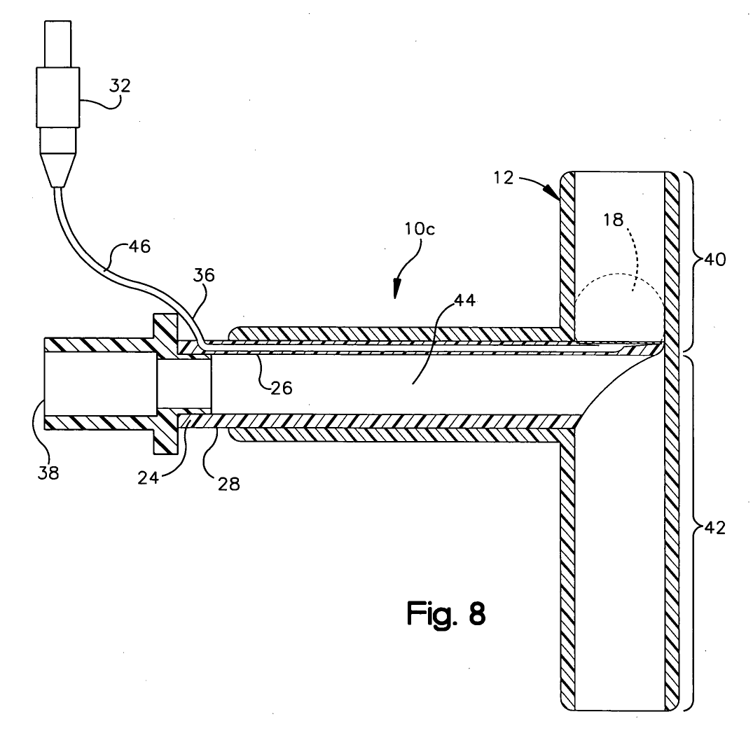

The tube 34 in the body 20, extends along the inner wall 26 of the body and includes a transverse portion 36 extending outwardly through the wall thickness 30 intermediate the distal and proximal ends 22, and 24. Alternatively, as shown in FIG. 8, the tube 34 can be disposed within the wall thickness 30 and include a transverse portion 36 extending outwardly through the wall thickness intermediate the distal and proximal ends 22, and 24.

The body 20_{c} and tube 34 can be made from any flexible, resiliently yieldable material which exhibits a substantially smooth, non-adherent surface. Examples of such material can include plastics (e.g., polyacrylics, polyolefins, and vinyl polymers such as polyvinyl chloride) and silicon. The material can also be transparent.

The body 20_{c} can also include a means for correctly orienting the apparatus 10_{c} relative to the T-tube 12. A key-and-channel configuration (not shown) may be used where, for example, the apparatus 10_{c} includes a channel which can be aligned with a key disposed in the first passage 14 of the T-tube 12. Alternatively, the apparatus 10C can include a key which is fitted to a channel disposed in the T-tube's first passage 14. Another means for correctly orienting the apparatus 10_{c} may include opposing markers (not shown) disposed about the apparatus and the T-tube 12. For instance, a colored marker may be placed on both the apparatus 10_{c} and the T-tube 12. When the apparatus 10_{c} is inserted into the T-tube 12, the markers on both the apparatus and the T-tube are aligned to ensure that the apparatus and the T-tube are properly oriented relative to each other.

As shown in FIG. 6, the inflatable device 18 is operatively secured to the distal end 22_{c} of the body 20_{c}. The inflatable device 18 can include any device, such as a balloon, capable of being inflated to a desired volume. The distal end 22_{c} of the body 20_{c} includes a recess 48 in which the inflatable device 18 is disposed when in its deflated position 18_{b}. The apparatus 10_{c} also includes a means 32 for selectively inflating and deflating the inflatable device 18. The means 32 can include a source of pressurized air, such as an air syringe, for example.

The inflatable device 18 may be inflated by the means 32. By forcing air from the means 32 through the lumen 46, the inflatable device 18 can be inflated 18ₐ to internally occlude the upper portion 40 of the second passage 16 and preclude air movement inwardly or outwardly through the upper portion. Alternatively, air can be evacuated through the lumen 46 to deflate the inflatable device 18 to the position 18_{b} in FIG. 6. In this case, the inflatable device 18 is disposed within the recess 48 in the outer surface of the distal end 22_{c} of the body 20_{c}. Withdrawing air from the lumen 46 clears the upper portion 40 of the second passage 16 and permits air flow in either vertical direction therethrough.

FIG. 7 is a cross-sectional illustration showing the apparatus 10_{c} when inserted into a T-tube 12. In this position, the apparatus 10_{c} can be used as a ventilator fitting by inflating the inflatable device 18 via the lumen 46 and means 32, and then inserting a ventilator adapter 38 into the open proximal end 24 of the apparatus. A mechanical ventilator (not shown) can then be connected to the ventilator adapter 38. Operation of the ventilator cyclically forces air inwardly through the body 20_{c}, downwardly through the lower portion 42 of the second passage 16, and into a patient's lungs. Importantly, since the inflated 18ₐ inflatable device 18 blocks the upper portion 40 of the second passage 16, nearly all of the inflowing air is downwardly directed into the patient's lungs. When the need for mechanical breathing assistance ceases, the inflatable device 18 can be deflated to the position 18_{b} in FIG. 6. Any minor volume lost due to air passing around the ventilator fitting can be compensated for by ventilator adjustments.

In addition, when the distal end 22_{c} of the apparatus 10_{c} is located in the second passage 16, the inflatable device 18 can be deflated 18_{b} and a suitable plug member (not shown) can be inserted into the open proximal end 24 of the apparatus. Insertion of the plug member permits the patient to breathe normally as the upper portion 40 of the second passage 16 is no longer occluded and air can pass upwardly and downwardly through the patient's trachea without escaping from the first passage 14 of the T-tube 12.

Alternatively, when the inflatable device 18 is deflated 18_{b}, the apparatus 10_{c} can be positioned such that the distal end 22_{c} is not positioned within the second passage 16. The plug member can then be inserted into the proximal end 24 of the apparatus 10_{c}. With the apparatus 10_{c} in this position, the patient can breathe normally as air can pass upwardly and downwardly through the patient's trachea.

Should the need arise to again provide the patient with mechanical breathing assistance, the apparatus 10_{c} can be rapidly converted to a ventilator fitting by removing the plug member, inserting the ventilator adapter 38 into the open proximal end 24 of the apparatus, and re-inflating the inflatable device 18 to again occlude the upper portion 40 of the second passage 16.

From the above description of the invention, those skilled in the art will perceive improvements, changes and modifications. Such improvements, changes and modifications within the skill of the art are intended to be covered by the appended claims.

Having described the invention, we claim:

1. An apparatus for use with a tracheal T-tube having transversely extending first and second passages that communicate with each other, said apparatus comprising:
a hollow body having a generally tubular shape with distal and proximal ends, said body having a channel extending between said distal and proximal ends, said body having cylindrical inner and outer walls that define a wall thickness, said body being insertable into the first passage of the T-tube so that said distal end lies in the second passage of the T-tube;
an inflatable device operatively secured to said distal end of said body; and
means for selectively inflating and deflating said inflatable device;
said body including a tube that defines a lumen extending between said ends and fluidly connecting said inflatable device with said means for selectively inflating and deflating said inflatable device.

2. The apparatus of claim 1, wherein said tube extends along said inner wall of said body.

3. The apparatus of claim 2, wherein said tube includes a transverse portion extending outwardly through said wall thickness intermediate said proximal and distal ends.

4. The apparatus of claim 1, wherein said tube is disposed within said wall thickness.

5. The apparatus of claim 4, wherein said tube includes a transverse portion extending outwardly through said wall thickness intermediate said proximal and distal ends.

6. The apparatus of claim 1, wherein said distal end of said body includes a recess in said cylindrical outer wall.

7. The apparatus of claim 1, wherein said distal end of said body has a planar shape and includes at least one opening oppositely located from said inflatable device.

8. The apparatus of claim 1, wherein said distal end of said body has an angled shape.

9. The apparatus of claim 1, wherein said inflatable device is disposed in said recess when said inflatable device is deflated.

10. The apparatus of claim 1, wherein said inflatable device is inflated by flowing pressurized air through said lumen.

11. The apparatus of claim 1, wherein said inflatable device is inflatable to occlude an upper portion of the second passage of the T-tube to preclude upward air flow therethrough, said inflatable device being deflatable to permit upward and downward air flow through said upper portion of said second passage.

12. An apparatus comprising:
a tracheal T-tube having transversely extending first and second passages that communicate with each other;
a hollow body having a generally tubular shape with distal and proximal ends, said body having a channel extending between said distal and proximal ends, said body having cylindrical inner and outer walls that define a wall thickness, said body being disposed in said first passage of said T-tube so that said distal end lies in said second passage of said T-tube;
means for selectively inflating and deflating said inflatable device; and
an inflatable device operatively secured to said distal end of said body;
said body including a tube that defines a lumen extending between said ends and fluidly connecting said inflatable device with said means for selectively inflating and deflating said inflatable device.

13. The apparatus of claim 12, wherein said tube extends along said inner wall of said body.

14. The apparatus of claim 13, wherein said tube includes a transverse portion extending outwardly through said wall thickness intermediate said proximal and distal ends.

15. The apparatus of claim 12, wherein said tube is disposed within said wall thickness.

16. The apparatus of claim 15, said tube includes a transverse portion extending outwardly through said wall thickness intermediate said proximal and distal ends.

17. The apparatus of claim 12, wherein said inflatable device is inflated by flowing pressurized air through said lumen.

18. The apparatus of claim 12, wherein said distal end of said body includes a recess in said cylindrical outer wall.

19. The apparatus of claim 12, wherein said distal end of said body has a planar shape and includes at least one opening oppositely located from said inflatable device.

20. The apparatus of claim 12, wherein said distal end of said body has an angled shape.

21. The apparatus of claim 12, wherein said inflatable device is disposed in said recess when said inflatable device is deflated.

22. The apparatus of claim 12, wherein said inflatable device is inflatable to occlude an upper portion of said second passage of said T-tube to preclude upward air flow therethrough, said inflatable device being deflatable to permit upward and downward air flow through said upper portion of said second passage.
